(19) United States
(12) Patent Application Publication
(10) Pub. No.: US 2007/0083190 A1
(43) Pub. Date: Apr. 12, 2007

(54) Compression device for a laser handpiece

(76) Inventor: Yacov Domankevitz, Newton, MA (US)

Correspondence Address: PROSKAUER ROSE LLP, ONE INTERNATIONAL PLACE 14TH FL, BOSTON, MA, 02110, US

(21) Appl. No.: 11/546,527

(22) Filed: Oct. 11, 2006

Related U.S. Application Data
(60) Provisional application No. 60/725,920, filed on Oct. 11, 2005.

(51) Int. Cl. A61B 18/18

(52) U.S. Cl. 606/9; 606/17

(57) ABSTRACT

A cosmetic condition (e.g., a pigmented lesion or a vascular lesion) can be treated using a delivery system that displaces blood from a target region of skin. The delivery system can include an optical element having a convex surface. The optical element can be a non-converging optical element that transmits the beam of radiation to the target region of skin.

### CROSS-REFERENCE TO RELATED APPLICATION

This application claims priority to and the benefit of U.S. provisional patent application Ser. No. 60/725,920 filed Oct. 11, 2005, the entire disclosure of which is herein incorporated by reference.

### FIELD OF THE INVENTION

The invention relates generally to using a beam of radiation and a compression device to treat a cosmetic condition, lesion, or disorder.

### BACKGROUND OF THE INVENTION

Pigmented lesions contain a light-absorbing chromophore, melanin, that has a broad absorption spectrum. Absorbance of light by melanin is strongest in the ultraviolet (UV) region of the electromagnetic spectrum and gradually diminishes toward the infrared region. Components of blood (e.g., hemoglobin, oxyhemoglobin, and methemoglobin) strongly absorb between 400 nm and 1,100 nm; therefore, extraneous light from a beam of radiation targeting a pigmented lesion in this wavelength region can be absorbed by one or more of these components of blood, resulting in unwanted side effects, such as purpura.

Furthermore, undesired purpura can also result during treatment of vascular lesions, such as leg veins or facial telangiectasias. The size of the pupura can be equal to the spot size of the incident beam, and it is common to use a laser spot size that is many times larger than the size of the targeted vessel. The purpura can result from the breaking of capillaries or other blood vessels above the targeted vessel. For example, when treating a 0.5 mm diameter vessel with a laser beam having a 7 mm diameter, purpura with a spot size of about 7 mm can result.

Kono treated facial lentigines using a long pulsed dye laser and a compression device. Kono et al., “Treatment of Facial Lentigines with the Long-Pulsed Dye Laser by Compression Method,” American Society for Laser Medicine and Surgery Abstracts, 33 (2004). A flat lens was attached to the tip of a laser handpiece to compress the skin and eliminate the absorption of light by oxyhemoglobin. A disadvantage of a flat lens, though, is that it does not uniformly displace blood. A flat lens can exert greater force around its periphery, and as a result, blood can pool in the central region of the lens.

U.S. Pat. No. 5,735,844 discloses a laser handpiece including a planoconvex lens for compressing the skin during a hair removal treatment. The planoconvex lens can be adapted to focus the beam of radiation below the surface of the skin, but if contact is not maintained between the skin and the lens while the beam of radiation is being delivered, the focal point of the radiation can change resulting in unwanted damage to the skin. For example, if the lens is withdrawn from the surface of the skin, the focal point of the lens can fall on the surface of the tissue causing a burn or resulting in a scar.

Therefore, what is needed, is a compression device that more uniformly displaces unwanted chromophores from a target region of skin to minimize unwanted side effects, such as purpura, and that is capable of providing a non-converging beam of radiation to reduce the risk of tissue damage resulting from burning or scarring when the compression device is delivering radiation while not in contact with the skin.

### SUMMARY OF THE INVENTION

The invention, in various embodiments, features a method and apparatus for delivering a beam of radiation to a target region of skin. The beam of radiation can be used to treat cosmetically pigmented and/or vascular lesions. The apparatus can include a compression device to displace unwanted chromophores from the target region. For example, a compression device can be used to displace blood from tissue in a target region while a beam of radiation targeting melanin is delivered to treat a pigmented lesion. In another example, a compression device can be used to displace blood from superficial capillaries while a beam of radiation targeting deeper blood vessels is used to treat an underlying vessel. An appropriate wavelength and pulse duration can be chosen to selectively damage or destroy the pigmented lesion with little or no injury to surrounding tissue. The compression device can include a negative focal length to diverge the beam of radiation to avoid unwanted damage to tissue surrounding the target region.

A treatment can include cooling to protect the skin surface, to minimize unwanted injury to the surface of the skin, and to minimize any pain that a patient may feel. An additional advantage of such a treatment according to the invention is that the treatment can be performed with minimal cosmetic disturbance such that the patient can return to normal activity immediately after the treatment.

Furthermore, the compression device can be used to treat blood vessels (e.g., varicose veins, telangiectasias, and reticular veins). The compression device can compress a targeted vessel. Compressing the vessel can reduce the diameter and/or change the shape of the targeted vessel so that radiation more uniformly irradiates the vessel. This can result in a more uniform heat distribution within the vessel and the vessel wall, and increase the efficiency of a treatment.

In one aspect, the invention features an apparatus for delivering a beam of radiation to a target region of skin. The apparatus includes a housing, an optical system disposed in the housing for delivering a beam of radiation to a target region of skin, and a meniscus lens disposed relative to a first end of the housing. The meniscus lens includes a convex surface in pressure contact with the target region of skin, and transmits the beam of radiation to the target region of skin.

In another aspect, the invention features an apparatus capable of treating a vascular lesion in a target region of skin. The apparatus includes a source generating a beam of radiation having a wavelength between about 400 nm and about 1,100 nm, and a delivery system remote from the source. The delivery system comprises an optical element including a convex surface. The optical element displaces blood from the target region, and transmits the beam of radiation to the target region of skin.

In yet another aspect, the invention features an apparatus capable of treating a pigmented lesion in a target region of skin. The apparatus includes a source generating a beam of radiation having a wavelength between about 400 nm and about 1,100 nm, and a delivery system remote from the source. The delivery system comprises an optical element including a convex surface. The optical element displaces blood from the target region, and transmits the beam of radiation to the target region of skin.

In still another aspect, the invention features a method of treating a vascular lesion in a target region of skin. The method includes placing an optical element having a convex surface adapted to contact the target region of skin, and applying pressure to the optical element to displace blood from the target region of skin. The beam of radiation is delivered to the target region of skin through the optical element to treat the vascular lesion in the target region of skin.

In another aspect, the invention features a method of treating a pigmented lesion in a target region of skin. The method includes placing an optical element having a convex surface adapted to contact the target region of skin, and applying pressure to the optical element to displace blood from the target region of skin. The beam of radiation is delivered to the target region of skin through the optical element to treat the pigmented lesion in the target region of skin.

In yet another aspect, the invention features an apparatus for contact cooling a target region of skin. The apparatus includes a housing, a transmitter of optical radiation into said housing, and a meniscus lens disposed on the housing in pressure contact with a surface of the target region of skin. The apparatus also includes an optical element and a cooling medium. The optical element is disposed on the housing and positioned between the transmitter and the meniscus lens, and the cooling medium passes through the housing and across a surface of the meniscus lens to cool the surface of the target region of skin below the temperature of the target region of skin.

In still another aspect, the invention features a method of delivering a beam of radiation to a target region of skin to treat a cosmetic condition. A meniscus lens disposed relative to a first end of a housing is provided. A convex surface of the meniscus lens is applied to a surface of the target region of skin. A beam of radiation is delivered through the meniscus lens to the target region of skin to treat the cosmetic condition.

Other aspects and advantages of the invention will become apparent from the following drawings and description, all of which illustrate the principles of the invention, by way of example only. In addition, although the embodiments are described primarily in the context of pigmented lesions and vascular lesions, other cosmetic conditions, lesions, and/or disorders can be treated using the invention. For example, treatments of hair, acne, wrinkles, skin laxity, blood vessels, fat, and cellulite are contemplated by the invention.

In various embodiments, the optical element can be a non-converging optical element. In some embodiment, the optical element can be a meniscus lens. In one embodiment, the meniscus lens is a negative lens that diverges the beam of radiation. In other embodiments, the meniscus lens can collimate the beam of radiation. In some embodiments, the optical element can be a planoconvex lens. In one embodiment, the optical element can include a first lens adapted to converge the beam of radiation and a second lens adapted to diverge the beam of radiation. The first lens can have a convex surface adapted to contact a surface of the target region of skin.

In some embodiments, an optical element can be used to substantially uniformly displace blood from a portion of the target region of skin (e.g., to minimize an unwanted side effect of a treatment). The blood displaced can be blood underlying a pigmented lesion, or overlying a vascular lesion. In various embodiments, a distance gauge can be disposed relative to the first end of the housing to position the housing spaced from the target region of skin. The distance gauge can define a hole for retaining the optical element and/or the meniscus lens.

Other aspects and advantages of the invention will become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows an apparatus for treating a cosmetic condition.

FIG. 2 shows profiles of volumetric heat production (J/cm³) of a blood vessels.

FIG. 3A shows a sectional view of a blood vessel in an uncompressed state. The figure was recorded using ultrasound imaging.

FIG. 3B shows a sectional view of the blood vessel shown in FIG. 3B in a compressed state.

FIG. 4 shows an embodiment of a system for treating a cosmetic condition.

FIG. 5A shows an apparatus that can be used to deliver a beam of radiation to a target region of skin to treat a cosmetic condition.

FIG. 5B shows an optical element that can be used with the apparatus shown in FIG. 5A.

FIG. 6 shows an apparatus capable of delivering a beam of radiation to a target region of skin to treat a cosmetic condition while cooling a surface of the target region of skin.

FIG. 7 shows a handpiece of an ultrasound device placed proximate to a skin surface.

### DESCRIPTION OF THE INVENTION

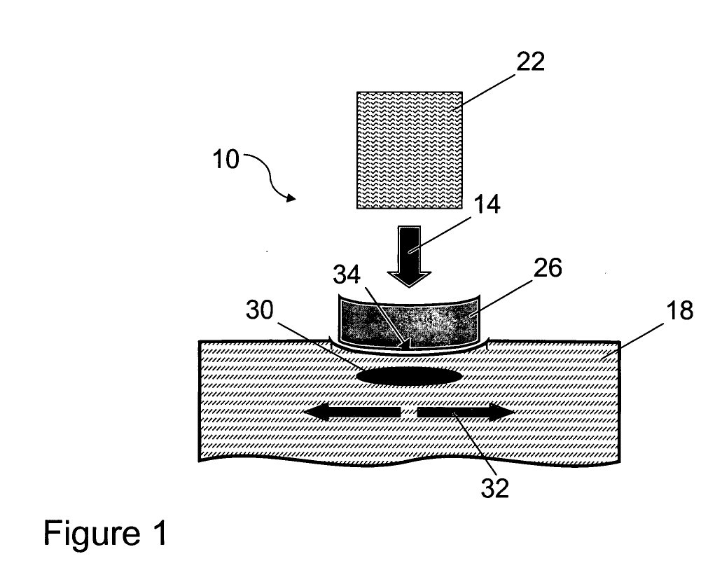

FIG. 1 shows an illustrative embodiment of an apparatus 10 delivering a beam of radiation 14 to a target region of skin 18. The apparatus includes a source 22 of the beam of radiation 14 and an optical element 26 for compressing the skin. The source 22 can generate the beam of radiation 14, or the source 22 can be a transmitter that delivers the beam of radiation to the target region of skin 18. For example, the transmitter can be an optical fiber or an optical waveguide. In other embodiments, the transmitter can include an articulated arm or an optical system that delivers a beam of radiation produced by a source through a system of lenses. Suitable optical elements 26 can include, but are not limited to, a lens and a plurality of lens. In the embodiment illustrated in FIG. 1, the optical element is a meniscus lens.

The target region of skin 18 can include a target feature 30, such as a pigmented lesion and/or a vascular lesion. In some embodiments, the beam of radiation 14 can be delivered to the target region of skin 18 to thermally injure, damage, and/or destroy a pigmented lesion and/or a vascular lesion. For example, the beam of radiation can be delivered to a target chromophore in the target region. Suitable target chromophores include, but are not limited to, melanin, melanin containing tissue, blood, hemoglobin, oxyhemoglobin, methemoglobin, and blood containing tissue.

Blood proximate to the target feature 30 can be displaced by pressing the optical element onto the skin surface. Displaced blood 32 can be overlying the target feature, underlying the target feature, in the target feature, contacting the target feature, adjacent the target feature, or a combination of the aforementioned. For example, for a superficial pigmented lesion, blood or a blood component underlying the target feature can be displaced so that radiation not absorbed by the pigmented lesion is absorbed or scattered by tissue other than the blood or blood component underlying the lesion. Absorption by blood or a blood component can result in unwanted injury such as purpura.

The optical element 26 can include a convex surface 34 contacting a surface of the skin 18. An optical element with a convex surface can substantially uniformly displace blood or a blood component by applying a substantially uniform force to the surface of the skin. Using a flat surface can result in blood pooling at various regions of the flat surface. For example, a flat surface can apply greater force around its periphery, and, as a result, blood can pool in a central region of the flat surface. In contrast, an optical element having a convex surface can displace blood uniformly because, when pressed against the skin, the optical element can contact the surface of the skin incrementally.

For example, in one embodiment, a central portion of the convex surface can contact the skin surface first as the optical element is brought into proximity of the surface of the skin. Blood can be displaced radially outward. An intermediate portion of the optical element can then come into contact with the surface of the skin displacing blood proximate to its point of contact, including blood displaced radially from the central portion. An outer portion of the optical element can then come into contact with the surface of the skin displacing blood proximate to its point of contact, including blood displaced radially from the central portion and the intermediate portion. If continuous pressure is applied, the blood can be precluded from diffusing radially inward to the central portion.

Furthermore, compression of the skin can bring the source of the beam of radiation into closer proximity to the target feature. Because the beam of radiation can be scattered, and thus attenuated, as it propagates through the skin, compression of the skin can result in more light reaching the target feature, which can increase the efficiency of a treatment. This can be advantageous for hair removal and for the treatment of cellulite, fatty tissue and acne, where the target feature tends to lie deeper in the skin. In one embodiment, the pressure applied to the optical element exceeds the blood pressure of the patient. For example, a whitening of the skin of the patient can be seen in the pressurized region when sufficient pressure is applied.

In various embodiments, the optical element can be a non-converging optical element. The optical element can diverge the beam of radiation, or the optical element can collimate the beam of radiation. The beam of radiation can be non-converging to prevent unwanted damage to the skin. For example, if contact between the optical element and the skin is not maintained during delivery of the beam of radiation, having a non-converging beam can preclude unwanted damage to the skin, which can result from focusing of the beam of radiation in or on the skin.

In various embodiments, the optical element is a meniscus lens; in other embodiments, the optical element is a planoconvex lens. The meniscus lens can have a negative focusing effect that can diverge the beam of radiation as it exits the meniscus lens and enters the skin. In one embodiment, the optical element is formed from a plurality of optical elements. For example, a first lens can be adapted to contact the skin surface, while a second lens is spaced from the first lens, positioned adjacent the first lens, or contacting the first lens. The first lens can converge the beam of radiation, and the second lens can diverge the beam of radiation. The sum of the two lenses can result in a beam of radiation that is collimated or that diverges as it exits the first lens and enters the skin.

In various embodiments, the optical element can be formed from a suitable optical material that is substantially transparent to the beam of radiation. Materials include, but are not limited to, quartz, BK7, fused silica, sapphire, an optical grade plastic, a biocompatible optical material, or a combination of the aforementioned. In various embodiments, the optical element can include an anti-reflective (AR) coating. The AR coating can be applied to the surface of the optical element not contacting the skin. In an embodiment in which the optical element includes two or more lens, a first lens, e.g., the lens contacting the surface of the skin, can include an AR coating on the surface of the lens not contacting the skin, and the second lens, e.g., the lens spaced from the surface of the skin, can include an AR coating on one or more surface of the lens.

In some embodiments, a compression device can be used to treat blood vessels (e.g., varicose veins, telangiectasias, and reticular veins). A blood vessel can be an artery, vein, or capillary. The compression device can include optical element 26 to compress a targeted vessel. Compressing the vessel can reduce the diameter of the vessel and/or change the shape of the targeted vessel so that radiation can more uniformly irradiate the vessel and its contents. In certain embodiments, the shape of the targeted vessel can be changed from substantially circular to substantially elliptical. Compressing the vessel can result in a more uniform heat distribution within the vessel and along vessel wall. For example, compressing the blood vessel can result in more energy penetrating to lower lying blood in the targeted vessel.

Radiation transmitted via the optical element 26 can irradiate blood or a component of blood within a targeted vessel. Radiation-induced vessel clearance can be based on selective photothermolysis. Heat can be transferred to the vessel wall at a temperature sufficient to thermally injure the vessel walls. In one embodiment, the vessel is irradiated to cause the vessel walls to be heated to at least 60° C. (e.g., between about 60° C. and about 100° C.). More uniform heating of the blood results in more uniform heat transfer to the vessel wall. After the vessel wall is heated, the vessel can undergo heat induced vessel contraction and/or intravascular thrombosis. Contraction results from direct heat induced collagen shrinkage and/or spasm. Intravascular thrombosis occurs after thermal denaturation of the inner vessel wall. The thermally damaged endothelium and perivascular tissue initiates a cascade of inflammation and wound healing, which can result in replacement of the vessel lumen by fibrous tissue. In some embodiments, vessel contraction and intravascular thrombosis occur simultaneously.

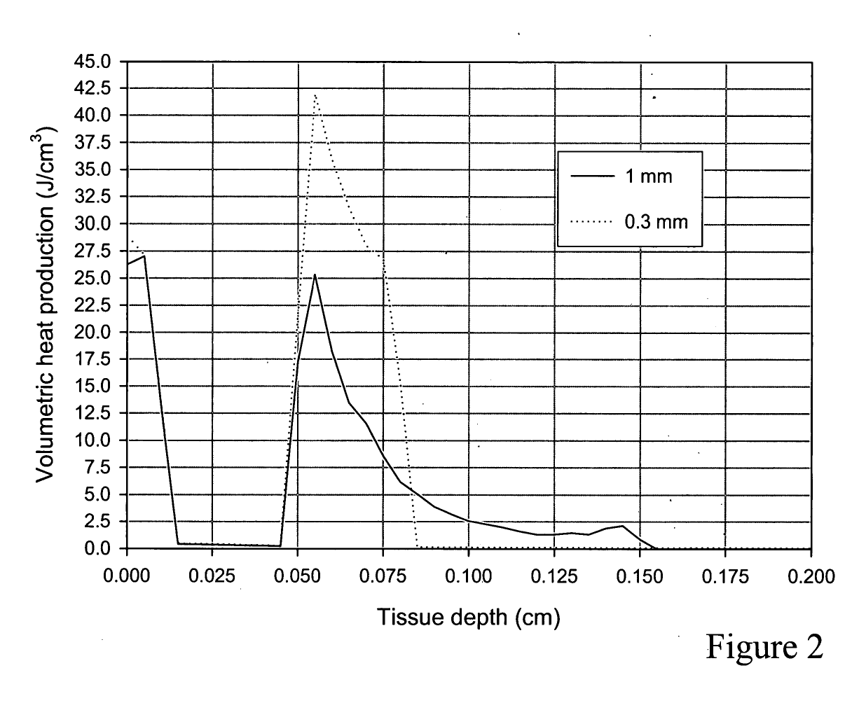

More uniform distribution of thermal heating can result in more effective and efficient radiation induced vessel clearance. FIG. 2 shows profiles of volumetric heat production (J/cm³) of a 1 mm vessel and a 0.3 mm vessel irradiated with a fluence of 1 J/cm² at 595 nm. The top surfaces of the vessels are about 0.5 mm below the surface of the skin. For the 1 mm vessel, the volumetric heat production decreases by about a factor of 10 from the top surface of the vessel to the bottom surface of the vessel (25 J/cm³ vs. 2.5 J/cm³). For the 0.3 mm vessel, energy is more evenly distributed throughout the entire vessel and the volumetric heat production decreases by only about a factor of 1.5 from the top surface of the vessel to the bottom surface of the vessel (42 J/cm³ vs. 27 J/cm³). Therefore, by compressing a blood vessel, radiation can be distributed more uniformly through the depth of the vessel.

In a vessel treatment according to the invention, a blood vessel is compressed and irradiated simultaneously or substantially simultaneously. The pressure applied is sufficient to cause the vessel to be compressed, but not enough to cause the skin (e.g., the epidermis or the dermis) to be substantially compressed. Therefore, the top surface of the blood vessel remains substantially the same with and without pressure being applied. That is, the target region of tissue is not substantially closer to the surface of the skin during a treatment.

The pressure applied also is not enough to entirely exclude blood from the target region and cause the vessel wall surfaces to contact and weld together. Typically, where the objective is to weld blood vessel walls together, the vessel walls are heated first, and then pressure is applied to cause the heated vessel wall surface to contact and weld together. If pressure is applied to cause the vessel wall surfaces to contact during irradiation, radiation passes through the vessel because a chromophore is not present to absorb the radiation.

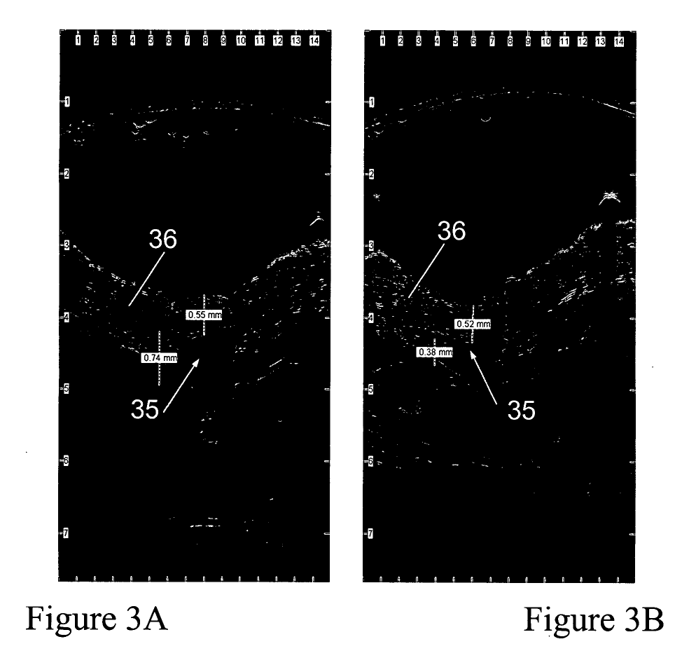

FIG. 3A shows an ultrasound image of a blood vessel 35 in an uncompressed state. The thickness of the skin 36 overlying the blood vessel 35 is about 0.55 mm. Blood vessel has a diameter of about 0.74 mm. FIG. 3B shows blood vessel 35 in a compressed state with a compressive pressure applied. The diameter of blood vessel 35 is reduced to 0.38 mm. The depth of the vessel did not vary significantly and is about 0.52 mm. As shown in FIG. 2, reducing the size of the target vessel improves uniformity of optical energy deposition and/or heat distribution along a vessel wall, which can result in improved vessel closure. It can also result in reduced side effects, such as purpura.

Furthermore, by compressing blood vessel, larger blood vessels can be targeted than, for example, using conventional means. For example, while a conventional Nd:YAG laser can be used to treat blood vessels no larger than about 2 mm, a vessel treatment according to the invention can be used on vessels up to about 4 mm, about 6 mm, about 8 mm, or about 10 mm. In certain embodiments, vessels between about 2 mm and about 10 mm can be treated. In some embodiments, vessels between about 2 mm and about 4 mm can be treated. In certain embodiments, vessels between about 4 mm and about 8 mm can be treated.

For pulsed dye lasers and frequency doubled Nd:YAG lasers, blood vessels no larger than 1.5 mm are typically treated. Using a compression device, blood vessels up to about 2 mm, about 3 mm, or about 4 mm can be treated. In certain embodiments, vessels between about 1.5 mm and about 4 mm can be treated. In some embodiments, vessels between about 1.5 mm and about 2 mm can be treated. In certain embodiments, vessels between about 2 mm and about 4 mm can be treated.

The spot size of the beam of radiation is typically larger than the diameter of the blood vessel. For example, a 1 mm blood vessel can be treated with a 3 mm beam of radiation. Blood vessels in a range of about 2 mm to about 4 mm can be treated with lasers having a spotsize of at least 6 mm. In certain embodiment, lasers having a spotsize up to 12 mm can be sued, although larger spotsizes can be used depending on the application. In some embodiments, a plurality of blood vessels are targeted by a beam of radiation.

Compression of a targeted vessel can be combined with a wavelength of radiation that is not strongly absorbed by blood or a blood component to improve uniformity of vessel heating. Furthermore, compression of the blood vessel can be effected by optical element 26, forced air, mechanical compression, hydraulic compression, pneumatic compression, or some combination of the aforementioned. In certain embodiments, the optical element 26 can have a flat surface contacting the skin surface.

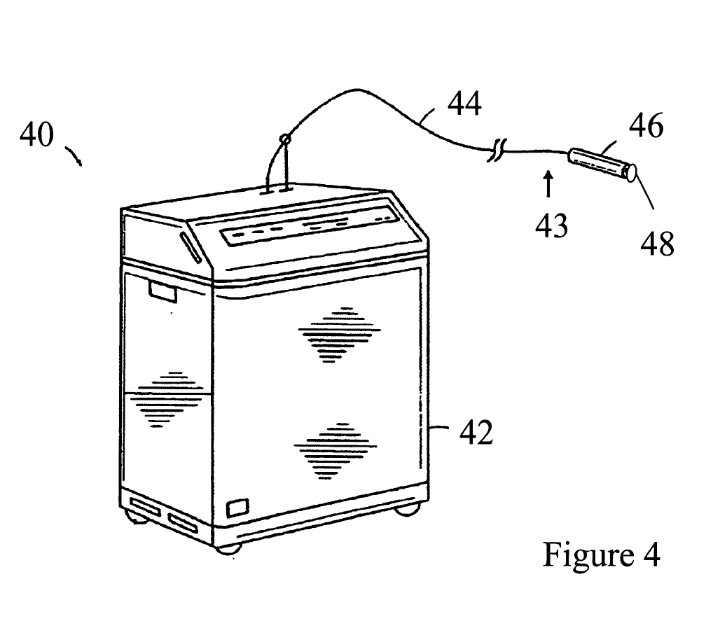

FIG. 4 shows an exemplary embodiment of a system 40 for treating tissue. The system 40 can be used to non-invasively deliver a beam of radiation to a target region. For example, the beam of radiation can be delivered through an external surface of skin over the target region. The system 40 includes an energy source 42 and a delivery system 43. In one embodiment, a beam of radiation provided by the energy source 42 is directed via the delivery system 43 to a target region. In the illustrated embodiment, the delivery system 43 includes a fiber 44 having a circular cross-section and a handpiece 46. A beam of radiation can be delivered by the fiber 44 to the handpiece 46, which can include an optical system (e.g., an optic or system of optics) to direct the beam of radiation to the target region. A user can hold or manipulate the handpiece 46 to irradiate the target region. The delivery system 43 can be positioned in contact with a skin surface, can be positioned adjacent a skin surface, can be positioned proximate a skin surface, can be positioned spaced from a skin surface, or a combination of the aforementioned. In the embodiment shown, the delivery system 43 includes a spacer 48 to space the delivery system 43 from the skin surface. In one embodiment, the spacer 48 can be a distance gauge, which can aid a practitioner with placement of the delivery system 43.

In various embodiments, the energy source 42 can be an incoherent light source or a coherent light source (e.g., a laser). Suitable laser include, but are not limited to, pulsed dye lasers, solid state lasers (e.g., Nd:YAG, Nd:YAP, alexandrite, KTP, and ruby lasers), diode lasers, and fiber lasers. In an embodiment using an incoherent light source or a coherent light source, the beam of radiation can be a pulsed beam, a scanned beam, or a gated continuous wave (CW) beam. The delivery system 43 can include a cooling apparatus for cooling an exposed surface of skin before, during, or after treatment.

In various embodiments, the beam of radiation can have a wavelength between about 200 nm and about 2,600 nm, although longer and shorter wavelengths can be used depending on the application. In some embodiments, the wavelength can be between about 200 nm and about 1,800 nm. In other embodiments, the wavelength can be between about 400 nm and about 1,100 nm. In some embodiments, the wavelength can be between about 1,100 nm and about 1,800 nm. In yet other embodiments, the wavelength can be between about 585 nm and about 600 nm. In some embodiments, the beam of radiation includes a band of wavelengths within a range. For example, the wavelength can be about 500-700 nm, 800-850 nm, 700-1100 nm, 930-1000 nm, 870-1400 nm, or 525-1200 nm. In certain embodiments, the beam of radiation includes a single wavelength from a range. For example, the wavelength can be about 532 nm, 585 nm, 595 nm, 630 nm, 694 nm, 755 nm, 830 nm, 1064 nm, or 1079 nm.

Exemplary pulsed dye lasers include V-Beam brand lasers and C-Beam brand lasers, both of which are available from Candela Corporation (Wayland, MA). Exemplary incoherent light sources include, but are not limited to, intense pulsed light sources, arc lamps, and flashlamps (e.g., an argon lamp, a xenon lamp, a krypton lamp, or a lamp that combines inert gases). An incoherent light source can include one or more filters to cutoff undesired wavelengths. For example, an ultra-violet filter (e.g., a filter that cuts off wavelengths less than about 350 nm) and/or a red or infra-red filter (e.g.,. a filter that cuts off wavelengths greater than about 700 nm) can be used together with an incoherent light source to provide a beam of radiation. An exemplary incoherent light source is an Ellipse system available from Danish Dermatologic Development A/S (Denmark).

In various embodiments, the beam of radiation can have a fluence between about 1 J/cm² and about 700 J/cm², although higher and lower fluences can be used depending on the application. In some embodiments, the fluence can be between about 10 J/cm² and about 150 J/cm². In one embodiment, the fluence is between about 5 J/cm² and about 100 J/cm². In certain embodiments, the fluence can be between about 10 J/cm² and about 50 J/cm². In some embodiments, the fluence can be between about 10 J/cm and about 20 J/cm². In one embodiment, the fluence is between about 1 J/cm² and about 10 J/cm². In one detailed embodiment, the fluence is about 1 J/cm², 10 J/cm², 15 J/cm², 20 J/cm², 25 J/cm², 50 J/cm², 100 J/cm², or 150 J/cm².

In various embodiments, the beam of radiation can have a spotsize between about 0.5 mm and about 25 mm, although larger and smaller spotsizes can be used depending on the application.

In various embodiments, the beam of radiation can have a pulsewidth between about 1 ns and about 30 s, although larger and smaller pulsewidths can be used depending on the application. In certain embodiments, the beam of radiation can have a pulsewidth between about 10 μs and about 30 s. In some embodiments, the beam of radiation can have a pulsewidth between about 1 ns and about 1 ms. In one embodiment, the beam of radiation can have a pulsewidth between about 0.45 ms and about 20 s. In one embodiment, the beam of radiation can have a pulsewidth between about 1 ms and about 1 s.

In various embodiments, the beam of radiation can be delivered at a rate of between about 0.1 pulse per second and about 10 pulses per second, although faster and slower pulse rates can be used depending on the application.

In various embodiments, the parameters of the radiation can be selected to deliver the beam of radiation to a predetermined depth. In some embodiments, the beam of radiation can be delivered to the target region up to about 10 mm below a surface of the skin, although shallower or deeper depths can be selected depending on the application. The predetermined depth can be 0.3 mm, 0.5 mm, 0.8 mm, 1 mm, 2 mm, 2.5 mm, 3 mm, 5 mm, 7 mm, or 10 mm.

In various embodiments, the tissue can be heated to a temperature of between about 50° C. and about 100° C., although higher and lower temperatures can be used depending on the application. In one embodiment, the temperature is between about 55° C. and about 70° C.

To minimize unwanted thermal injury to tissue not targeted (e.g., an exposed surface of the target region and/or the epidermal layer), the delivery system 43 shown in FIG. 4 can include a cooling system for cooling before, during or after delivery of radiation, or a combination of the aforementioned. Cooling can include contact conduction cooling, evaporative spray cooling, convective air flow cooling, or a combination of the aforementioned. In one embodiment, the handpiece 46 includes a skin contacting portion that can be brought into contact with the skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device can be used to cool the skin (e.g., a DCD available from Candela Corporation). For example, the delivery system 43 shown in FIG. 4 can include tubing for delivering a cooling fluid to the handpiece 46. The tubing can be connected to a container of a low boiling point fluid, and the handpiece can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon or tetrafluoroethane.

In various embodiments, a gel can be applied to the skin. The gel can facilitate matching the index of refraction between the skin and the optical element 26. In certain embodiments, better thermal contact between the optical element 26 and the skin can be achieved. In an embodiment where the optical element 26 is translated across the skin during a treatment, the gel can assist a practitioner with smoothly sliding the optical element 26.

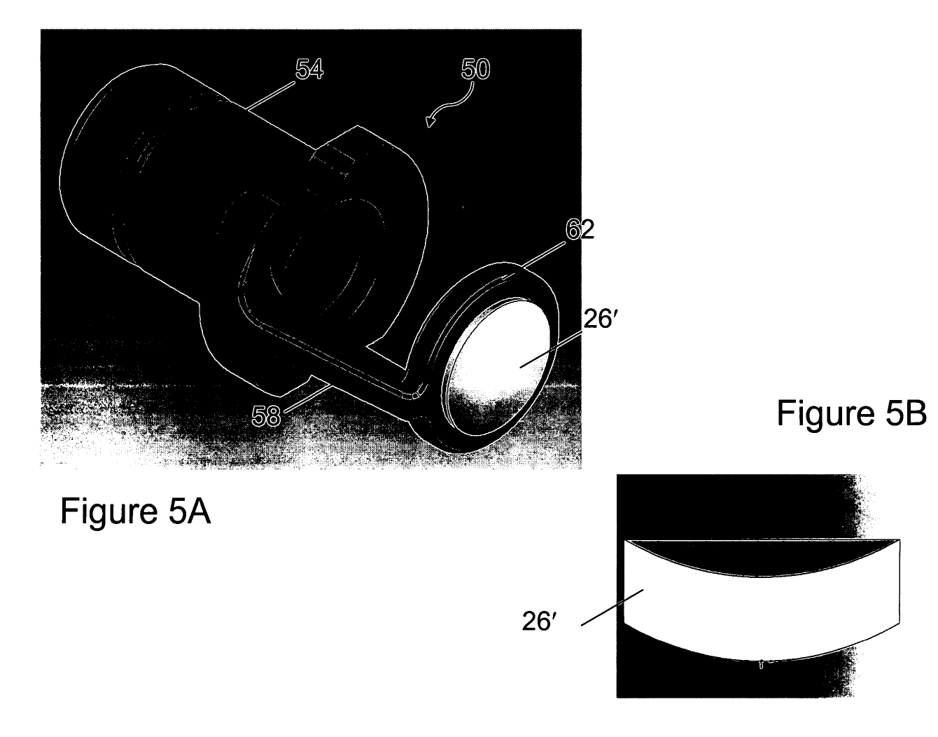

FIG. 5A shows an illustrative embodiment of an apparatus 50 that can be used to deliver a beam of radiation to a target region of skin. The apparatus can include a housing 54 and a distance gauge 58 for positioning the apparatus 50 relative to the skin. In one embodiment, the housing 54 can seat over the handpiece 46 shown in FIG. 4. In one embodiment, the housing 54 can be the handpiece 46. The distance gauge 58 can include a ring portion 62 affixed to the end of the distance gauge 58 or formed as part of the distance gauge 58. The ring portion 62 can define a hole or an aperture that retains optical element 26′. As shown in FIG. 5B, optical element 26′ is a meniscus lens.

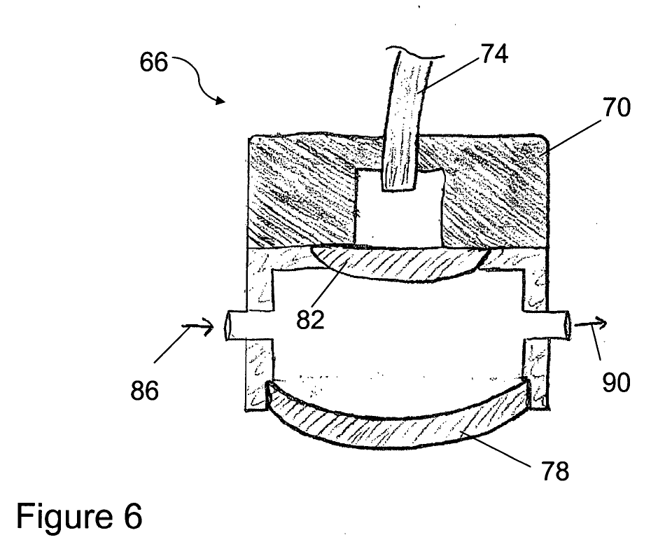

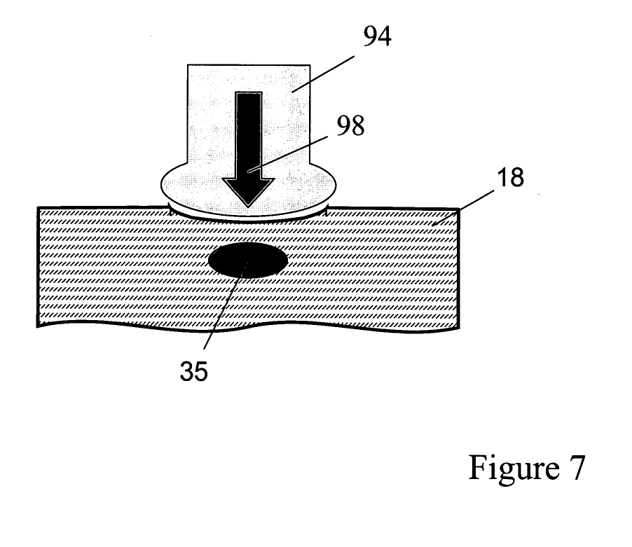

FIG. 6 shows an illustrative embodiment of an apparatus 66 that can be used to deliver a beam of radiation to a target region of skin and is capable of cooling a surface of the target region of skin. The apparatus 66 includes a housing 70, a transmitter 74 of radiation, a first lens 78, a second lens 82, and a path for a cooling medium. The housing 70 includes an inlet 86 and an outlet 90 to allow a cooling medium to flow across a surface of the first lens 78. The apparatus 66 can be used in the treatment of various cosmetic conditions, lesions, and/or disorders such as pigmented lesions, vascular lesions, blood vessels, hair, acne, wrinkles, skin laxity, skin discolorations, and fat.

The transmitter 74 of radiation can be an optical fiber or other optical waveguide. The first lens 78 can be a meniscus lens. The second lens 82 can be a planoconvex lens or other suitable lens. In various embodiments, the sum of the focusing effect of the first lens 78 and the second lens 82 can result in a beam of radiation that is non converging as it exits the first lens 78. For example, the beam of radiation can be diverging or collimated as it exits the first lens 78 and enters the skin.

The cooling medium can be a cooling fluid that is substantially optically transparent to the beam of radiation. For example, the cooling medium can be water or nitrogen gas, although other suitable cooling mediums can be used. Alternatively, the housing 74 can include an electrically controlled cooler (e.g., thermoelectric cooled, Stirling cooled, or Peltier cooled). In various embodiments, the apparatus 66 can maintain the temperature of a surface or an upper portion of the skin between about −15° C. and about 20° C.

In various embodiments, an ultrasound device can be used to measure depth or position of a blood vessel to be targeted. For example, a high frequency ultrasound device can be used. A handpiece of an ultrasound device can be placed proximate to the skin to make a measurement. In one embodiment, the ultrasound device can be place in contact with the skin surface. The ultrasound device can deliver ultrasonic energy to measure position of the blood vessel or the shape of the blood vessel.

In certain embodiments, a single handpiece 94 can be used to deliver ultrasonic energy and the beam of treatment radiation. The handpiece 94 can compress the skin 18 while delivering the beam of radiation 98 to the targeted blood vessel 35.

The time duration of the cooling and of the radiation application can be adjusted so as to maximize the thermal injury to the vicinity of the target region. For example, if the position of a target feature is known, then parameters of the optical radiation, such as pulse duration and/or fluence, can be optimized for a particular treatment. Cooling parameters, such as cooling time and/or delay between a cooling and irradiation, can also be optimized for a particular treatment. Accordingly, a zone of thermal treatment can be predetermined and/or controlled based on parameters selected.

While the invention has been particularly shown and described with reference to specific illustrative embodiments, it should be understood that various changes in form and detail may be made without departing from the spirit and scope of the invention.

What is claimed:

1. An apparatus for delivering a beam of radiation to a target region of skin, comprising:
a housing;
an optical system disposed in the housing for delivering a beam of radiation to a target region of skin; and
a meniscus lens disposed relative to a first end of the housing and having a convex surface in pressure contact with the target region of skin, the meniscus lens transmitting the beam of radiation to the target region of skin.

2. The apparatus of claim 1 wherein the meniscus lens comprises a negative lens that is adapted to diverge the beam of radiation.

3. The apparatus of claim 1 wherein the meniscus lens is adapted to collimate the beam of radiation.

4. The apparatus of claim 1 wherein the wavelength of the beam of radiation is between about 400 nm and about 1,100 nm.

5. The apparatus of claim 1 further comprising a distance gauge disposed relative to the first end of the housing to position the housing spaced from the target region of skin.

6. The apparatus of claim 5 wherein the distance gauge defines a hole for retaining the meniscus lens.

7. The apparatus of claim 1 wherein the meniscus lens substantially uniformly displaces blood from a portion of the target region of skin.

8. An apparatus for treating a pigmented lesion in a target region of skin, comprising:
a source generating a beam of radiation having a wavelength between about 400 nm and about 1,100 nm; and
a delivery system remote from the source, the delivery system comprising an optical element including a convex surface that displaces blood from the target region, the optical element transmitting the beam of radiation to the target region of skin.

9. The apparatus of claim 8 wherein the optical element comprises a non-converging optical element.

10. The apparatus of claim 8 wherein the optical element comprises a meniscus lens.

11. The apparatus of claim 8 wherein the optical element comprises a planoconvex lens.

12. The apparatus of claim 9 wherein the optical element comprises:
a first lens having a convex surface contacting a surface of the target region of skin, the first lens adapted to converge the beam of radiation; and
a second lens adapted to diverge the beam of radiation.

13. The apparatus of claim 8 wherein the delivery system comprises a distance gauge disposed relative to an end of the delivery system to position the delivery system spaced from the target region of skin.

14. The apparatus of claim 11 wherein the distance gauge defines a hole for retaining the optical element.

15. The apparatus of claim 8 wherein the optical element substantially uniformly displaces blood from the target region of skin.

16. An apparatus for treating a vascular lesion in a target region of skin, comprising:
a source generating a beam of radiation having a wavelength between about 400 nm and about 1,100 nm;
a delivery system remote from the source comprising an optical element including a convex surface that displaces blood from the target region, the optical element transmitting the beam of radiation to the target region of skin.

17. The apparatus of claim 16 wherein the optical element comprises a non-converging optical element.

18. The apparatus of claim 16 wherein the optical element comprises a meniscus lens.

19. The apparatus of claim 16 wherein the optical element comprises a planoconvex lens.

20. The apparatus of claim 17 wherein the optical element comprises:
a first lens having a convex surface contacting a surface of the target region of skin, the first lens adapted to converge the beam of radiation; and
a second lens adapted to diverge the beam of radiation.

21. The apparatus of claim 15 wherein the delivery system comprises a distance gauge disposed relative to an end of the delivery system to position the delivery system spaced from the target region of skin.

22. The apparatus of claim 18 wherein the distance gauge defines a hole for retaining the optical element.

23. The apparatus of claim 15 wherein the optical element substantially uniformly displaces blood from the target region of skin.

24. A method of treating a pigmented lesion in a target region of skin, comprising:
contacting to the target region of skin an optical element having a convex surface;
applying pressure to the optical element to displace blood from the target region of skin; and
delivering a beam of radiation to the target region of skin through the optical element to treat the pigmented lesion in the target region of skin.

25. The method of claim 24 wherein the blood displaced underlies the pigmented lesion.

26. The method of claim 24 further comprising displacing blood from the target region of skin to minimize an unwanted side effect of a treatment.

27. The method of claim 24 wherein the optical element comprises a non-converging optical element.

28. The method of claim 24 wherein the optical element comprises a meniscus lens.

29. The method of claim 27 wherein the optical element comprises:
a first lens having a convex surface contacting a surface of the target region of skin, the first lens adapted to converge the beam of radiation; and
a second lens adapted to diverge the beam of radiation.

30. The method of claim 24 further comprising diverging the beam of radiation with the optical element.

31. The method of claim 24 wherein the optical element substantially uniformly displaces blood from the target region of skin.

32. A method of treating a vascular lesion in a target region of skin, comprising:
contacting to the target region of skin an optical element having a convex surface;
applying pressure to the optical element to displace blood from the target region of skin; and
delivering a beam of radiation to the target region of skin through the optical element to treat the vascular lesion in the target region of skin.

33. The method of claim 32 wherein the blood displaced overlies the vascular lesion.

34. The method of claim 32 further comprising displacing blood from the target region of skin to minimize an unwanted side effect of a treatment.

35. The method of claim 32 wherein the optical element comprises a non-converging optical element.

36. The method of claim 32 wherein the optical element comprises a meniscus lens.

37. The method of claim 35 wherein the optical element comprises:
a first lens having a convex surface contacting a surface of the target region of skin, the first lens adapted to converge the beam of radiation; and
a second lens adapted to diverge the beam of radiation.

38. The method of claim 32 further comprising diverging the beam of radiation with the optical element.

39. The method of claim 32 wherein the optical element substantially uniformly displaces blood from the target region of skin.

40. An apparatus for contact cooling a target region of skin, comprising:
a housing;
a transmitter of optical radiation into said housing;
a meniscus lens disposed on the housing and adapted to be in pressure contact with a surface of the target region of skin;
a lens disposed on the housing and positioned between the transmitter and the meniscus lens; and
a cooling medium passing through the housing and across a surface of the meniscus lens to cool the surface of the target region of skin below the temperature of the target region of skin.

41. A method of delivering a beam of radiation to a target region of skin to treat a cosmetic condition, comprising:
providing a meniscus lens disposed relative to a first end of a housing;
applying a convex surface of the meniscus lens to a surface of the target region of skin; and
delivering a beam of radiation through the meniscus lens to the target region of skin to treat the cosmetic condition.
